(19) United States
(12) Patent Application Publication
(10) Pub. No.: US 2021/0161379 A1
(43) Pub. Date: Jun. 3, 2021

(54) SCANNING LASER OPHTHALMOSCOPES

(71) Applicant: Oxford University Innovation Limited, Oxford (GB)

(72) Inventors: Laura YOUNG, Oxford (Oxfordshire) (GB); Timothy MORRIS, Durham (Durham) (GB); Hannah SMITHSON, Oxford (Oxfordshire) (GB); Christopher SAUNTER, Durham (Durham) (GB)

(21) Appl. No.: 17/261,184

(22) PCT Filed: Jul. 19, 2019
(86) PCT No.: PCT/GB2019/052024

§ 371 Date: Jan. 19, 2021

(30) Foreign Application Priority Data
Jul. 20, 2018 (GB) 1811903.2

(51) Int. Cl. A61B 3/10; A61B 3/14

(52) U.S. Cl. CPC A61B 3/1025; A61B 3/14

(57) ABSTRACT

A scanning laser ophthalmoscope comprises a light source (LS) arranged to generate a beam of light; a deformable mirror relay (DM, SM1); a first scanning relay (VS, SM2); a second scanning relay (HS, SM3); a wavefront sensor (WFS); and a detector (APD). The deformable mirror relay comprises a deformable flat mirror (DM) arranged to compensate for distortions detected by the wavefront sensor (WFS). Each of the scanning relays comprises a flat scanning mirror (VS, HS) arranged to scan the beam across the eye of a subject. At least one of the relays has an entry focal plane and an exit focal plane and further comprises a spherical mirror arranged to reflect the beam from the entry focal plane onto the flat mirror and to reflect light from the flat mirror into the exit focal plane.

### FIELD OF THE INVENTION

The invention relates to scanning laser ophthalmoscopes and in particular to adaptive optics scanning laser ophthalmoscopes. It also has application in other systems that relay pupil conjugate planes, such as scanning confocal microscopes.

### BACKGROUND TO THE INVENTION

A scanning laser ophthalmoscope (SLO) generally comprises a light source that generates a light beam, two scanners, which may use movable mirrors, to scan the beam in orthogonal directions across the part of the eye to be imaged, and a detector, such as a photodiode, to measure the reflected beam in order to generate image data.

Optical aberrations in the eye degrade the quality of the focused beam on the first pass and further degrade it as the light is reflected back out of the eye. A confocal pinhole in the SLO acts to spatially filter the reflected light to improve the effective resolution, but with a reduction in the amount of light reaching the detector. A larger pinhole can be used to improve the throughput but at the expense of both lateral and axial resolution.

The adaptive optics scanning laser ophthalmoscope (AOSLO) further adds to the system a wavefront sensor for sensing optical distortions, and a deformable mirror or other optical element to modify the illuminating light beam to correct for these detected distortions. The AOSLO was first developed in 2002 and since then the technology has been adopted in several laboratories around the world, for clinical and vision science research.

The first use of adaptive optics (AO) to improve the resolution and throughput of a scanning laser ophthalmoscope by producing a high-quality focus at the retina and at the pinhole was achieved by Roorda et al in 2002, making imaging of individual cones close to the fovea possible. In the time since, AOSLO has become important for studying the anatomy and morphology of the retinal components at a cellular scale. Images obtained with an AOSLO give in vivo structural information such as the arrangement and density of the photoreceptors as well as the size, composition and arrangement of the blood vessels. Further developments have opened up the possibility of studying other retinal structures such as inner segments of cones, imaged via split-detection, the retinal pigment epithelium, imaged via dark-field techniques and blood vessels and retinal ganglion cells imaged via offset-aperture imaging. AOSLO imaging is increasingly being used to study the function of the retina, such as using single-cell stimulation to understand the neural wiring in the retina, measuring intrinsic responses to stimulation, monitoring blood flow and retinal activity, and analyzing motion distortions within the images for eye tracking with high spatial and temporal resolution.

To date, reflective AOSLOs used around the world have been built broadly to one of three designs. The first generation AOSLO (Roorda et al., 2002; Roorda et al., 2005) used tilted spherical mirrors on-axis to create a series of 4-f relays, re-imaging the pupil of the eye on to horizontal and vertical scanners as well as on to a deformable mirror (DM). The use of mirrors, rather than lenses, eliminates back reflections that would otherwise contaminate the wavefront sensor image, avoids chromatic aberration, and gives a higher throughput. However, using spherical mirrors in this way introduces astigmatism into the wavefront and this accumulates with each reflection from a spherical mirror. Later, Dubra and Sulai (Dubra & Sulai, 2011) and Merino et al. (Merino et al., 2011) developed a second-generation AOSLO that balanced astigmatism in one axis with astigmatism in the perpendicular axis by using an out-of-plane design in which the optical path is not contained in a plane parallel to the optical bench. These systems were more compact and removed the need for an astigmatic correction lens by reducing the astigmatic errors to a simple focus term. This led to a diffraction-limited field of view of 1 degree at a wavelength of 450 nm. In 2013 Liu et al. (Liu et al., 2013) developed an in-plane AO-OCT (adaptive optics optical coherence tomography) design that avoided the introduction of astigmatism by using toroidal, rather than spherical mirrors. This system achieves diffraction-limited performance within 1.8 degrees at 800 nm. However, toroidal mirrors require custom specification and manufacture, and are expensive.

Although astigmatism can be avoided by using lenses rather than spherical mirrors, lenses have traditionally been avoided when designing AOSLOs. Back reflections from lenses can affect image quality and wavefront sensor performance and can be comparable in intensity to the signal from the retina. In 2012, Felberer et al. (Felberer Liu et al., 2012) developed a lens-based system that uses a pair of quarter-wave plates to remove back reflections from the optics. This allowed a more compact system to be developed with a larger theoretical diffraction-limited field of view (good image quality is achieved with a 4 degree field of view at a wavelength of 840 nm). However, lens-based systems are still limited due to chromatic aberration and wavelength-dependent polarization effects. Additionally, a lens-based system relies on the polarization of light being unaltered by the sample, which may not be the case when light is scattered (e.g. in retinal pigment epithelium cell imaging) or when imaging fluorescence emission.

One goal of the AOSLO design is to achieve diffraction-limited imaging using a compact instrument. Two issues in the commercialization of AO retinal imaging techniques are cost and the space required for such instruments.

### SUMMARY OF THE INVENTION

The present invention provides an adaptive optics scanning laser ophthalmoscope, or other scanning confocal microscope, comprising: a light source arranged to generate a beam of light; a deformable mirror relay; a first scanning relay; a second scanning relay; a wavefront sensor and a detector. Each of the scanning relays may comprise a flat scanning mirror arranged to scan the beam across the eye of a subject. At least one of the relays has an entry focal plane and an exit focal plane and further comprises a spherical mirror arranged to reflect the beam from the entry focal plane onto the flat mirror and to reflect light from the flat mirror into the exit focal plane.

The scope may further define a pupil plane, at which the pupil of the eye to be examined should be located. The pupil plane may be conjugated with at least one of the deformable mirror and the scanning mirrors, and may be conjugated with all three.

Said one of the relays may be the deformable mirror relay. The deformable mirror relay may comprise a deformable flat mirror arranged to compensate for distortions detected by the wavefront sensor. The deformable mirror relay may further comprise a first fold mirror between the entry focal plane and the spherical mirror and/or a second fold mirror between the spherical mirror and the exit focal plane. This can allow the entry focal plane and/or the exit focal plane to not be coplanar with the deformable mirror. This may be useful for packaging the system as the deformable mirror may be housed in a relatively large unit.

Alternatively said one of the relays may be one of the scanning relays.

Indeed, each of the relays may have an entry focal plane and an exit focal plane and further comprise a spherical mirror arranged to reflect the beam from the respective entry focal plane onto the respective flat mirror and to reflect light from the respective flat mirror into the respective exit focal plane.

The relays may be ‘in plane’ i.e. coplanar. For example the light beam within all three relays may, for at least one orientation of each of the scanning mirrors, remain in one plane.

At least two of the relays may be arranged in respective modules, which can be assembled together for use. The modules may be configurable in a plurality of different operable configurations.

Each of the modules may have an entrance focal plane and an exit focal plane. The ophthalmoscope may include connecting means, arranged to connect the modules together so that one of the focal planes of one of the modules is coincident with one of the focal planes of the other of the modules.

Ophthalmoscopes according to the invention use a reflective, rather than refractive, design that carries additional benefits where broadband or multi-wavelength light sources are used and avoid back reflections that can affect image quality and wavefront sensor performance.

Ophthalmoscopes according to some embodiments of the invention can maintain a planar optical alignment without the build-up of astigmatism using compact, reconfigurable modules based on an Offner relay system. This design can result in a compact system that is simple to align and, being composed of modular relays, has the potential for additional components to be added. Such systems can maintain diffraction-limited image quality across the field of view whereby cones are resolved in both the peripheral and the central retina. The modular relay design is generally applicable to any system requiring one or more components in the pupil conjugate plane. This is likely to be useful for any point-scanned system, such as a standard scanning laser ophthalmoscope or other confocal imaging system.

Ophthalmoscopes according to some embodiments of the invention may additionally use a digital oscilloscope for data capture.

The AOSLO design may be in-plane, simplifying the construction and alignment. It may comprise three (or more) configurable fully-reflective pupil relay modules, one for each scanning mirror and one for the DM. Such a design could in principle also be used for other types of scanned light microscopy, where pupil conjugate planes must be re-imaged.

The AOSLO may further comprise, in any combination, any one or more features of the embodiments of the invention, which are shown in the accompanying drawings, as will now be described.

### BRIEF DESCRIPTION OF THE DRAWINGS

FIG. 1 is a schematic diagram of an AOSLO according to an embodiment of the invention;

FIG. 2 shows the fraction of light transmitted through the AOSLO of FIG. 1 to the eye as a function of pupil shift and the fraction of light enclosed as a function of radius from the centroid at the retina;

FIG. 3 shows the effect of the parameters of the relays in the AOSLO of FIG. 1 on the quality of the image;

FIG. 4 shows the point spread function of the light beam at various points in the relays of the system of FIG. 1;

FIG. 5 is a plan view of the two scanning relays of the AOSLO of FIG. 1;

FIG. 6 shows how the PSF varies with scan angle of the two scanning mirrors in the AOSLO of FIG. 1; and

FIG. 7 shows the effect of the adaptive optics components in the AOSLO of FIG. 1.

### DESCRIPTION OF THE PREFERRED EMBODIMENTS

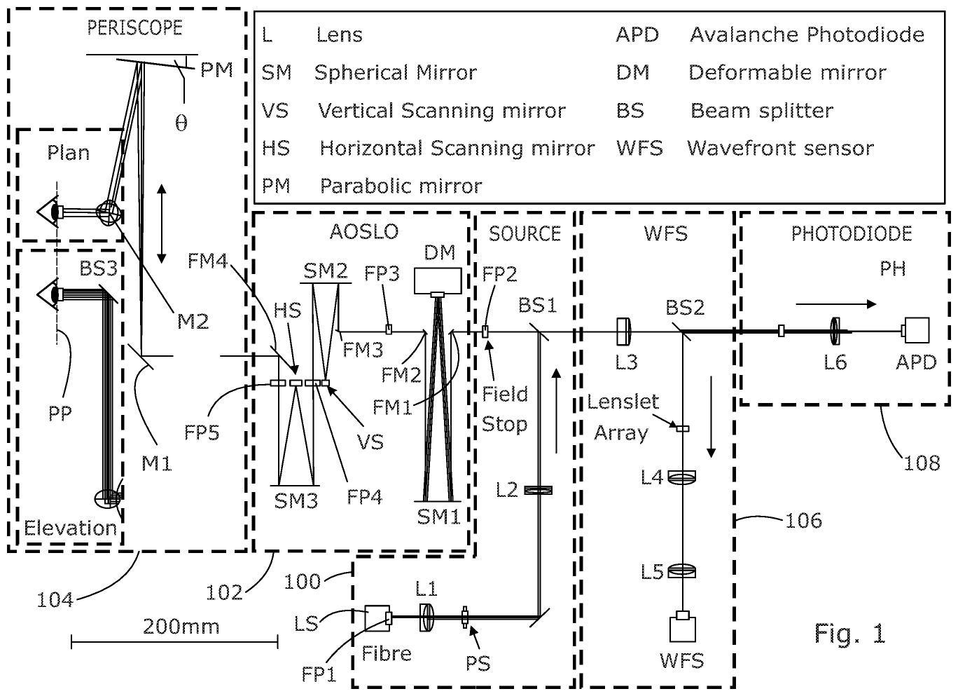

Referring to FIG. 1, the AOSLO comprises a number of components, which may conveniently be provided in a number of modules. For example, these may include a light source module 100, an AOSLO or adaptive optics scanning module 102, a periscope module 104, a wavefront sensing module 106 and a detector or photodiode module 108. The source module 100 relays the light beam from a superluminescent diode (fibre-fed) via reflection (e.g. 8% reflection) from a beam splitter BS1 to the AOSLO module 102, where the scanning and wavefront compensation takes place. The scanned, wavefront-compensated light is delivered to the eye of a subject via the periscope module 104, which defines a pupil plane PP where the pupil of the eye is to be located for inspection, and light returning from the eye passes back through the periscope and AOSLO modules 102. The beamsplitter BS1 in the source module 100 transmits a proportion (e.g. 92%) of the returned light into the next two modules 106, 108. A proportion (e.g. 8%) of that transmitted light is reflected into the WFS module 106, where the wavefront is sensed. The remaining proportion (e.g. 92%) of the light is transmitted by beamsplitter BS2 in the WFS module 106 to the photodiode module 108, where the light is detected. Light propagation direction within modules is indicated by the arrows.

While the optical layout of the system is shown in FIG. 1 as separated into five functional modules, the system can be divided up or configured differently as circumstances require.

In the following description the optical design of each module is provided, followed by an analysis of end-to-end system performance of the embodiment as shown in FIG. 1. System optical performance was modeled using the Zemax optical design software. Unless stated otherwise, for the purposes of image scaling, a paraxial model eye was used in the modelling with a pupil diameter of 7 mm and a focal length of 17 mm.

Source Module

The light source module 100 comprises a light source LS, a collimating lens L1 and a pupil stop PS, a further lens L2 and a beamsplitter BS1. The light source LS used may, for example, be a fiber-coupled 850 nm (50 nm FWHM) superluminescent diode (BLMS-mini-351-HP3-SM-OI, SuperLum). The output fiber (P5-780A-PCAPC-1, Thorlabs) may have a mode-field diameter of 5 μm with a numerical aperture of 0.12. The lens L1, for example of focal length 40 mm (AC254-40-B-ML, Thorlabs) may be used to collimate the light from the light source so that the projected image of the fiber at the model retina (corresponding to the expected position of the retina of the eye being scanned in use) has a nominal diameter of 2.1 μm.

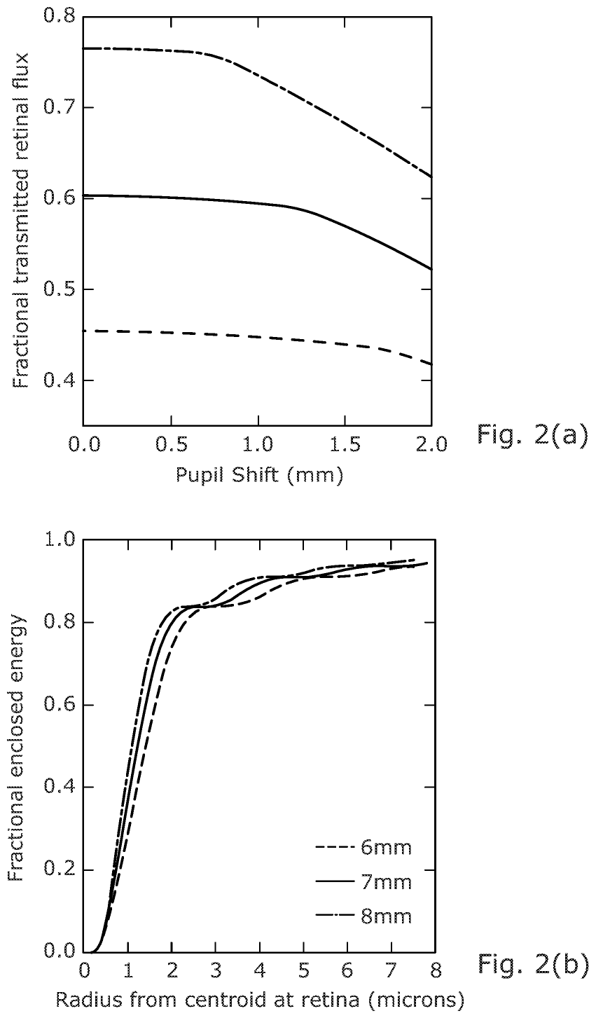

FIG. 2(a) shows the fraction of transmitted flux at the eye pupil (i.e. in the pupil plane PP) compared to flux after the pupil stop PS which forms the source aperture, with offset of the eye pupil from the center of the Gaussian intensity profile for 6, 7 and 8 mm diameter eye pupils. The change in gradient at large pupil offsets for 7 and 8 mm diameter pupils corresponds to the pupil moving outside the 9.4 mm diameter illuminated area of the eye. FIG. 2(b) shows modelled on-axis fractional enclosed energy for the 5 μm diameter source fiber projected onto the retina for eye pupil diameters of 6 to 8 mm at 850 nm.

After the collimating lens L1, a circular aperture (the pupil stop) conjugated to the eye pupil is used to define a beam of the desired (e.g. 4 mm) diameter. A 4 mm aperture corresponds, in the embodiment shown, to a projected diameter of 9.4 mm at the eye pupil that has a Gaussian intensity profile with a 1/e² diameter of 21.8 mm. Motion of the pupil within this intensity profile causes a variation in flux at the retina, as modelled in FIG. 2. For a 7 mm diameter pupil, up to 1.2 mm of pupil motion is possible before vignetting of the pupil occurs. In practice, a bite-bar and dental impression can be used during imaging and the pupil position can be expected to be stable to within 1 mm, resulting in a flux variation of up to 1.5%.

The final elements in the source module are a 200 mm focal length lens L2 (e.g. AC254-200-B, Thorlabs) creating a telecentric output beam with an f-ratio of 49.4. A beamsplitter BS1 (e.g. a 92/8 beamsplitter CP1-BP108, Thorlabs) reflects a proportion, e.g. 8%, of the light from the fiber into the system. A variable aperture field stop is placed at the focal plane (FP2) after the beamsplitter BS1 to reject any stray and scattered light returned through the system, as well as provide an alignment reference target. The diameter of this aperture is much larger than the diffraction limit and therefore does not impact the measured wavefront.

The functional light path from the source LS to the beamsplitter BS1 is in one direction only (although there will typically be a small amount of reflected light travelling in the opposite direction). Between the beam splitter BS1 and the focal plane FP2 light passes in both directions, towards and away from the eye (i.e. the pupil plane). It will be noted that the exit beam from the source module 100 towards the AOSLO module 102, and the other exit beam from the source module 100 towards the detector module 108 are parallel to each other, and may indeed, as in the embodiment shown, be coincident with each other, exiting the module 100 on opposite sides of the module in opposite directions. This makes assembling the modular system simple.

The AO system only corrects for aberrations present between the retina and the wavefront sensor. There are two sources of non-common path optical aberrations: between the wavefront sensor WFS and the detector pinhole PH and between the light source and the beamsplitter BS1. One source of non-common path aberrations can be calibrated however both cannot be simultaneously corrected with a single deformable mirror. Residual aberrations in the source path increase the size of the illuminated patch at the retina and increase the size of the wavefront sensor spots and the size of the focal spot at the pinhole PH. Residual aberrations between the wavefront sensor and the pinhole PH effectively lower the flux reaching the detector.

AOSLO Module

The AOSLO module contains a series of reflective spherical mirror modules. Each of these modules is in the form of a modified Offner relay and comprises a spherical mirror and one of the scanning mirrors SM2, SM2 or the deformable mirror DM. Light travels through the whole of the AOSLO module 102, i.e. through all three of the relays, in both directions, from the source module 100 to the periscope module 104, and back from the periscope module 104 to the source module 100 after reflection from the eye. The relay modules can be placed in any order, a typical arrangement is to place the deformable mirror module closest to the light source, followed by the vertical scanning mirror and then the horizontal scanning mirror.

One of the relays is a deformable mirror relay and comprises a deformable mirror DM and a spherical mirror SM1. The deformable mirror may for example be a 3.5 μm stroke MultiDM from Boston Micromachines. The spherical mirror SM1 reflects light from the source module 100 onto the DM, which reflects it back to the spherical mirror SM1 which re-focuses it at a focal plane FP3. The optical path length between the focal plane FP2 and the spherical mirror SM1, is equal to the optical path length between the spherical mirror SM1 and the focal plane FP3, and also equal to the optical path length between the deformable mirror DM and the spherical mirror SM1.

However, a fold mirror FM1, FM2 may be provided between each of the focal planes FP2, FP3 and the spherical mirror SM1 each arranged to turn the beam through 90° so that the focal planes FP2 and FP3 are parallel to each other and both at 90° to the centre of the spherical mirror SM1 and the DM. This allows the two focal planes FP2, FR3 to be non-coplanar with the deformable mirror DM, which in turn allows the deformable mirror relay to reflect at very narrow angles regardless of the physical size of the deformable mirror DM. All of the components of the relay, i.e. the deformable mirror and the spherical mirror and, if present, one or two fold mirrors, may be mounted on a common support so that they form a deformable mirror module.

It will be noted that the entrance beam of the deformable mirror relay, which is incident on the spherical mirror SM1, from the input focal plane FP2, in this case via the fold mirror FM1, and the exit beam of the deformable mirror relay, which is the reflected beam from the spherical mirror SM1 towards the exit focal plane FP3, in this case via the fold mirror FM2, are always parallel to each other.

Another of the relays is a scanning mirror relay, in this case a vertical scanning mirror relay, and comprises a first scanning mirror VS and a spherical mirror SM2. The spherical mirror SM2 reflects light from the exit focal plane of the deformable mirror relay FP3 onto the vertical scanning mirror VS, which reflects it back to the spherical mirror SM2 which re-focuses it at a focal plane FP4. The optical path length between the focal plane FP3 and the spherical mirror SM2, is equal to the optical path length between the spherical mirror SM2 and the focal plane FP4, and also equal to the optical path length between the vertical scanning mirror VS and the spherical mirror SM2. All of the components of the vertical scanning relay, i.e. the vertical scanning mirror and the spherical mirror SM2 and, if present, one or two fold mirrors, may be mounted on a common support so that they form a vertical scanning mirror module.

Again, it will be noted that the entrance beam of the vertical scanning mirror relay, which is incident on the spherical mirror SM2, from the input focal plane FP3, in this case via the fold mirror FM3, and the exit beam of the vertical scanning mirror relay, which is the reflected beam from the spherical mirror SM2 towards the exit focal plane FP4, in this case directly with no fold mirror, are always parallel to each other.

The third of the relays is a second scanning mirror relay, in this case a horizontal scanning mirror relay, and comprises a second scanning mirror HS and a spherical mirror SM3. The spherical mirror SM3 reflects light from the exit focal plane of the vertical scanning relay FP4 onto the horizontal scanning mirror HS, which reflects it back to the spherical mirror SM3 which re-focuses it at a focal plane FPS. The optical path length between the focal plane FP4 and the spherical mirror SM3, is equal to the optical path length between the spherical mirror SM3 and the focal plane FP5, and also equal to the optical path length between the horizontal scanning mirror HS and the spherical mirror SM3. All of the components of the relay, i.e. the horizontal scanning mirror and the spherical mirror SM3 and, if present, one or two fold mirrors, may be mounted on a common support so that they form a horizontal scanning mirror module. Again, it will be noted that the entrance beam of the horizontal scanning mirror relay, which is incident on the spherical mirror SM3, from the input focal plane FP4, in this case directly with no fold mirror, and the exit beam of the horizontal scanning mirror relay, which is the reflected beam from the spherical mirror SM3 towards the exit focal plane FP5, in this case directly with no fold mirror, are always parallel to each other.

It will further be noted that, in the arrangement shown in FIG. 1, the entrance and exit beams of the horizontal scanning relay are parallel to the entrance and exit beams of the vertical scanning relay. Furthermore, in each case, the entrance and exit beams are also parallel to the entrance and exit beams of the deformable mirror relay. This makes it relatively simple for the three relay modules to be rearranged in a different order, which may be desirable in some cases. It is of course a requirement that in whatever sequence the relays are arranged, the exit focal plane of the first relay in the sequence is coincident with the entrance focal plane of the second relay in the sequence (at FP3 in the arrangement of FIG. 1), and the exit focal plane of the second relay in the sequence is coincident with the entrance focal plane of the third relay in the sequence (at FP4 in the arrangement of FIG. 1). If further relays are required, these can be added into the sequence of relays, as further modules, again with correct alignment of the respective focal planes. The relay modules may be connectable together using clips or fastenings, or may each be removably mounted on a common support, such as a board or rack, which connects them together in the various configurations.

An example of an additional relay is a gross focus relay, in which the element at the pupil plane would be a focus-correcting element such as a deformable mirror. A system that corrects the higher and lower order (focus and astigmatism) aberrations separately is referred to as a ‘woofer-tweeter’ system. Other examples include: additional wavefront correcting elements to perform multiconjugate AO for widefield correction; a steering system to stabilise the position of the eye pupil; and amplitude modulation in pupil, for example to simulate or null a spatially varying opacity such as a cataract. These may be incorporated into the AOSLO module 102. However the modular structure of the relays allows additional relay modules or components to be inserted into the AOSLO between any two of the relay modules shown or at the beginning or end of the chain of relays.

With the use of fold mirrors (or in other arrangements without them) the entrance and exit beams to the whole AOSLO module 102 may be parallel to each other, and may also be on opposite sides of the module. This makes assembling a modular system, including the AOSLO module, simple.

The two scanning mirrors VS, HS are flat. The deformable mirror DM is typically formed of a membrane that can be deformed using a number of movable actuators but deformable mirrors can also be formed of a number of moveable mirror sections. The movable mirror surface can be aligned so that it lies in a single flat plane so that the deformable mirror is flat, but parts of the surface are independently movable out of that flat plane so as to deform the mirror from its flat configuration.

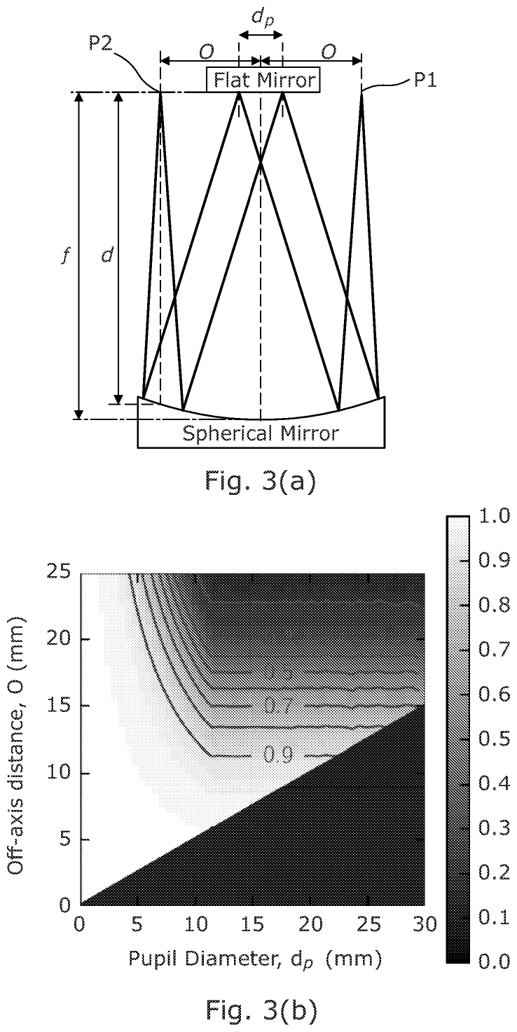

Each of the relays creates a pupil-conjugate plane, i.e. a plane that is conjugate with the pupil plane PP. These are at the locations of the deformable mirror DM (which may for example be a Boston Micromachines MultiDM) and each of the scanning mirrors (which may for example be a Electro-Optical Products Corporation PLD-XYG, SC-30 raster scanning system). Each of these three pupil conjugate relays is based upon the Offner relay as described in U.S. Pat. No. 3,748,015. An Offner relay re-images an input focal plane with 1:1 magnification using two concentric spherical mirrors. Here the convex mirror of the true Offner relay is replaced with a flat mirror (either the DM or one of the scanning mirrors VS, HS). This reduces the optical performance of the system compared to the true Offner relay. However diffraction-limited performance can still be achieved if the combination of operating wavelength, pupil diameter, focal length and off-axis distance remains within a limited parameter space, as described in more detail below with reference to FIG. 3.

Referring to FIG. 3a, each of the three relays comprises a concave spherical mirror of focal length f and a flat mirror facing each other along a common optical axis X. As described above, the deformable mirror is flat when it is in its flat configuration, which is assumed in FIG. 3. The light path through the relay is shown from point P1 in the focal plane FP (corresponding to FP2 and FP3 in the deformable mirror relay for example) on one side of the flat mirror, onto the spherical mirror, then onto the flat mirror, back to the spherical mirror and then back to a point P2 in the focal plane on the other side of the flat mirror. The pupil diameter at the flat mirror is dₚ, and the offset distance of the points P1 and P2 from the optical axis of the spherical mirror is O. dₚ is set by the pupil stop in the source module 100. The magnification between this stop and each of the pupil conjugates then changes the physical size of the pupil at these conjugate locations. The size of the pupil stop is chosen such that the smallest mirror (the vertical scanning mirror VS) is filled and the pupil there is not clipped by the edges of that scanning mirror. Points P1 and P2 are the positions of the beam at the entry to and exit from the relay, i.e. the centre of the beam in focal planes FP2 and FP3 in the DM relay, the focal planes FP2 and FP3 of the VS relay and the focal planes FP3 and FP4 of the HS relay, in each case assuming that the folding mirrors are not present. The distance d between each of the points P1 and P2 and the spherical mirror, in the direction parallel to the optical axis of the relay X, depends on the offset distance O, and tends towards f as O decreases.

FIG. 3(b) shows how the Strehl ratio, which is a measure of the quality of optical image formation, varies with pupil diameter and offset distance for a re-imaged 850 nm point source using a 200 mm focal length spherical relay. The solid black area indicates the region that is physically impossible since the off-axis distance O cannot be less than half of the diameter dₚ of the pupil. A Strehl ratio of 1 describes zero wavefront error. It can be seen that for smaller pupil diameters, a high Strehl ratio can be obtained with a wide range of values of the distance O. However for larger pupil diameters dₚ, the reduction in Strehl ratio becomes significant, but the effect can be minimized by reducing the off-axis distance O towards the minimum. It will be appreciated that the values of Strehl ratio are also dependent on the focal length of the spherical mirror and the wavelength of the reflected light.

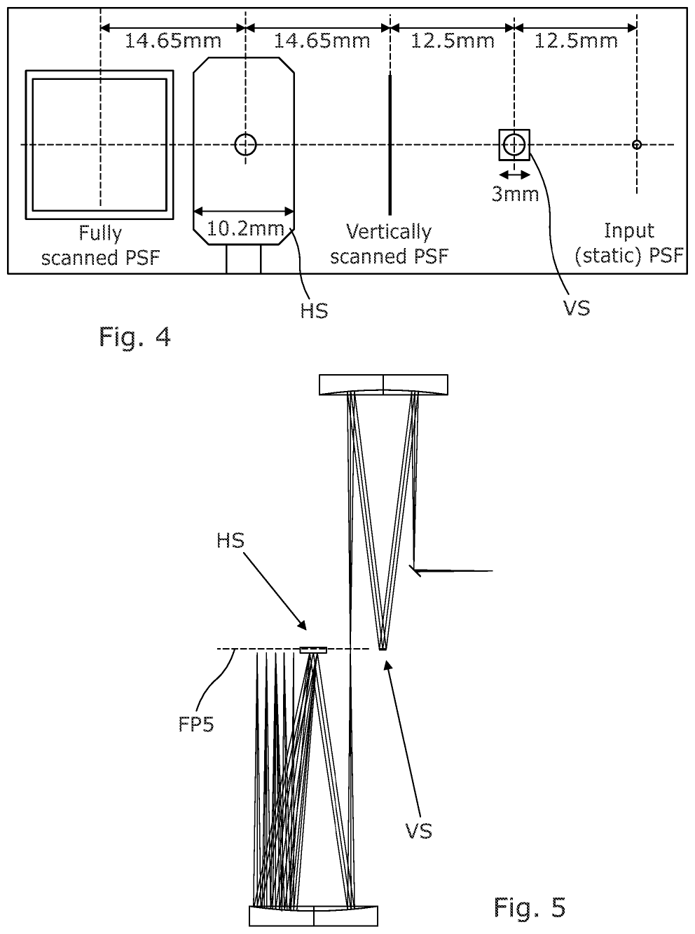

FIG. 4 shows the configuration of the scanning mirrors VS, HS in the plane of the scanning mirrors from input unscanned focal point to the output scanned focal plane. Beam diameters at the scanning mirrors are shown by the circles on the VS and HS.

FIG. 5 is a plan view and optical path through scanning mirrors, showing how the path varies for different field positions that relate to different scan angles of the horizontal scanning mirror HS.

The parameters of each relay are constrained by the size of each of its components. In the embodiment shown, wavefront compensation is achieved using a square geometry 140 actuator, 400 μm pitch DM (MultiDM, Boston Micromachines). The active area of the DM is 4.2 mm square, requiring a 200 mm focal length spherical mirror relay (SM1 is a CM508-200-E02, Thorlabs) to give the correct magnification at the eye pupil. As the DM enclosure is wider than the spherical mirror relay, two D-shaped 5 mm diameter fold mirrors (Thorlabs PFD05-03-P01) FM are used to direct the input and output beams away from it. The double-pass path through the protective window of the DM does not impact optical performance at a level that would be observable in the complete system. To ensure collimation after reflection from the spherical mirror the axial position of the spherical mirror must be adjusted compared to the nominal focal length to take into account the surface curvature and off-axis distance. Referring back to FIG. 3, the corrected distance between mirror and focal plane, d, is given by

d=(f²-O²)^{1/2}

where f is the focal length of the spherical mirror, and O is the off-axis distance as defined in FIG. 3(a). For the 12.5 mm off-axis distance used in the DM relay, d=199.6 mm.

In the embodiment shown, the pupil diameter at both the fast (vertical) and slow (horizontal) scanning mirrors is 2.1 mm, produced using 100 mm focal length spherical mirror (SM2/SM3: Thorlabs CM508-100-E02) relays. The optical layout of the scanning system of FIG. 1 is defined by the requirement to avoid vignetting of the scanned output beam by the final spherical mirror SM3. The scanning system in this configuration has maximum scan angle of 2° the slow (vertical at the eye) axis, corresponding a section of the retina approximately 0.6 mm in width (dependent upon the optical properties of the eye). In practice, the fast scanner has a maximum scan angle of 1° and, to achieve equal sampling density in the horizontal and vertical axes, the slow scan angle is reduced to 1.6°.

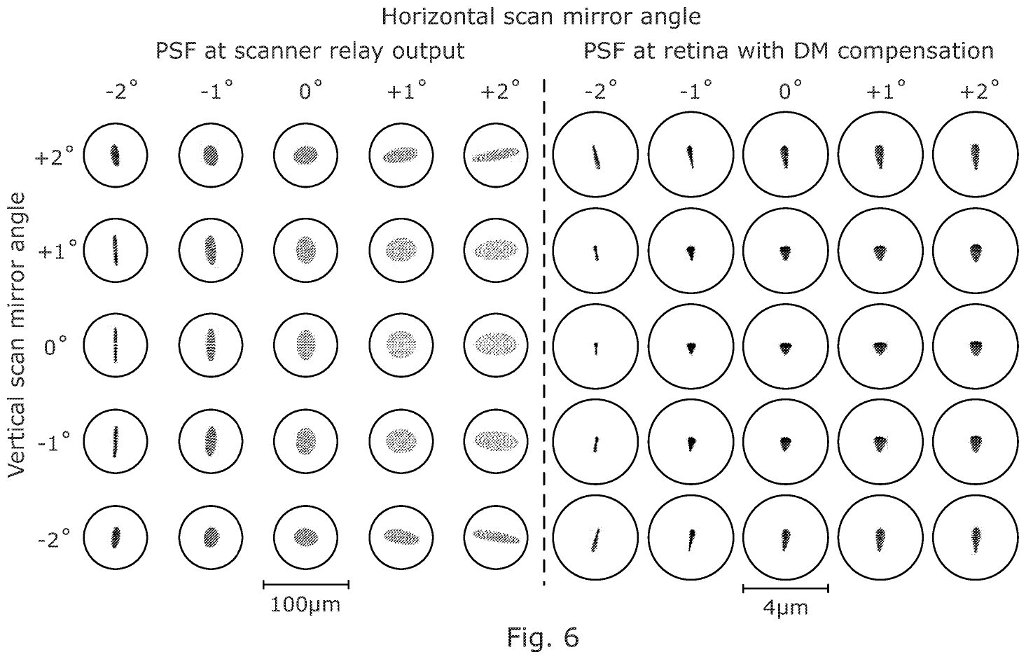

The optical path through the scanning mirror relay is not static, and therefore aberrations across the field of view defined by the scan angle can vary. FIG. 6 shows a ray-trace through the scanning relay (left side) and at the retina of the model eye (right side) showing PSF every degree over the 2° scanning mirror range compared to the diffraction-limited Airy diameter at 850 nm (black circles). Common aberrations present at the retina have been compensated using the deformable mirror. The 2° horizontal scan angle is the field point closest to the slow scanning (horizontal) mirror.

At each field point, the ray-trace remains within the Airy diameter and therefore the system remains diffraction-limited over this scanning range at this wavelength. Note that the diffraction limit referred to within FIG. 6 describes the diffraction limit of the maximum pupil diameter (fully illuminated DM) and does not take into account the smaller pupil diameter of the eye. The scanned field has a mean image scale of 0.278 mm per degree of scan at the model-eye retinal plane. Optical aberrations can lead to some anamorphism in the reconstructed image. The optical design predicts a maximum deviation of the position of the PSF from the expected position on the retina of 2.4 μm. Mean deviation is 0.71 μm with a standard deviation of 0.47 μm. We correct for this a posteriori through calibration using a grid target placed in the scanned focal plane (e.g. Edmund Optics 62-209).

Periscope

The functions of the periscope are to create a collimated beam with an accessible DM/scanning mirror conjugate plane (pupil plane PP) at which the participant's pupil can be placed, and to include a beamsplitter that separates the 850 nm AOSLO light from visible wavelengths. This allows the participant to be shown stimuli, such as fixation targets, during experiments. One advantage of the in-plane AOSLO relay design is that a 100 mm height periscope can raise the beamsplitter (BS3: DMSP805L, Thorlabs) above the main optical system and provide a wide field of view for fixation targets and other psychophysical stimuli.

A refractive relay may be used in the periscope, however back reflections from the long focal length lens are bright compared to the signal backscattered from the retina, so this is not preferred. A reflective relay is used in the periscope of the system shown using an off-axis segment of a 444 mm focal length parabolic mirror (PM: Edmund Optics 32-064-566), as well as a first fold mirror M1, directing the beam on to the parabolic mirror PM and a second fold mirror M2, which directs the beam upwards to a dichroic beamsplitter BS3. The 850 nm imaging light is directed into the eye via the dichroic beamsplitter BS3. This provides a 9.35 mm diameter collimated beam at the pupil plane PP positioned 444 mm from the parabolic mirror PM. For optimal alignment of an off-axis parabolic mirror, the off-axis distance and mirror tilt angle θ are related by

θ=arctan(O/f)

For the 76.2 mm diameter parabolic mirror the maximum off-axis distance that reflects the scanned field without vignetting is 25 mm, defining a 3.22° tilt angle. This shallow angle does not provide sufficient space to separate input and output beams, therefore the angle must be increased to allow for the periscope optomechanics. The DM can be used to compensate for any optical aberrations that are common across the field at the expense of DM stroke. Including this degree of freedom allows the parabolic mirror tilt angle θ to be increased to 7° at an off-axis distance of 14.4 mm. This compensates for the majority of field-dependent aberrations present within the scanning system. Whilst the relay output remains diffraction-limited under these conditions with a residual RMS wavefront error across the full scanned region of better than λ/10, the addition of a small astigmatic term on the DM (peak-to-valley amplitude on the DM surface of 0.11 μm) can also be applied to correct for common aberrations. This corresponds to 6.2% of the DM mechanical stroke and results in a RMS wavefront error across the full scanned region of the retina of better than λ/50 at wavelength λ of 850 nm.

The limited stroke of the DM used in the current embodiment allows for a limited range of focus correction. In the embodiment shown ophthalmic trial lenses are used to correct for large focus errors. Gross focus compensation and correction of large optical errors can be integrated with an additional optical relay that includes a large stroke DM, tuneable lens or other correcting element such as a spatial light modulator, or with the addition of a Badal lens system.

Wavefront Sensor

The wavefront sensing (WFS) module 106 comprises a lens L3 and a beamsplitter BS2. On entering the WFS module 106 light in the entrance beam to the WFS module, returning from the eye passes through the lens L3 and is then split by the beamsplitter BS2. A proportion, in the embodiment shown 8%, of the return flux from the eye is directed towards the wavefront sensor WFS after transmission through BS1 in the source module 100 and reflection from BS2. In this embodiment, the wavefront sensor (WFS) relay optics re-image the 12×12 actuator DM surface onto 11×11 lenslets of a 300 μm pitch, 5.1 mm focal length lenslet array (18-00211, SUSS micro-optics) using a 150 mm focal length lens (L3: Thorlabs AC254-150-B). This results in each sub-aperture of the wavefront sensor corresponding to 0.9×0.9 mm at the eye pupil. Optical distortion between the lenslet array and DM is less than 0.1%, which ensures the lenslet array remains aligned to the DM in the optimal Fried geometry.

The resulting lenslet spot pattern is then re-imaged onto the CCD (Edmund Optics EO-0312M) using a 1:1 refractive relay comprising two matched 40 mm focal length achromatic lenses (L4 and L5: Thorlabs AC254-040-B). Achromatic lenses were used within this design because they provided superior imaging performance across the field compared to singlet lenses of similar focal length. An additional benefit of the refractive WFS relay is that it creates an accessible DM conjugate plane in which the WFS camera can be placed during alignment. This enables accurate conjugation of the lenslet array to the DM plane. The relay also simplifies optomechanical mounting because the lenslet array need not be placed within 5 mm of the detector. The designed WFS centroid response after the relay across a 2° input field angle remains linear to better than 1%.

The FWHM of the optimal retinal PSF within each sub-aperture is 8.123 μm, or 0.821 pixels. The WFS is not confocal, therefore sensing operates on light backscattered from the retina that is not limited to a single plane. For a scattering depth of 40 μm defocus within the WFS increases the PSF FWHM to >1 pixel, avoiding sub-sampling effects within the WFS that can cause non-linearities in WFS response.

Photodiode Module

The photodiode module receives the collimated exit beam from the WFS module after BS2 (84% of the return flux present at the field stop). In the current embodiment, a 125 mm focal length lens (L6: Thorlabs AC254-125-B) focuses light onto one of a set of pinholes that range in size from 20 to 300 μm in diameter. We can estimate that a 89.9 μm diameter pinhole is required to enclose 50% of the flux from the retina.

Adaptive Optics System

Adaptive optics correction is achieved, in this embodiment, using a microelectromechanical (MEMS) DM and a custom-made Shack-Hartmann wavefront sensor.

Each of the modules 100, 102, 104, 106, 108 may have its own housing, or at least support structure, as represented by the broken lines in FIG. 1. Also each of the source 100, AOSLO 102 and WFS 106 modules has two entry or exit apertures through which the beam can enter or exit the module. The photodiode module 108 only has one entry aperture through which the beam can enter the module. The periscope 104 has one entry/exit aperture though which the beam can enter traveling towards the eye and exit travelling away from the eye. Each of the modules may have fewer or more components than those shown in FIG. 1. For example the AOSLO module may comprise one or more further relays as described above.

In the embodiment shown, real-time control was achieved using custom code, written in the Python programming language and the Numpy multidimensional array library. Before an imaging session an interaction matrix is generated, mapping deformable mirror actuator voltages to spot motion in the wavefront sensor using a 2.4× scale model eye. The control matrix is calculated as the pseudo-inverse of this matrix using a singular value decomposition. The reconditioning value used in the singular value decomposition was selected as that giving the best closed-loop stability on testing. On each cycle of the closed loop, intensity thresholding is used to define the sub-apertures that should be included in the calculation of the actuator voltages, allowing for some movement of the eye pupil. The center of mass of the image from each sub-aperture is calculated, from which the null spot positions are subtracted, to measure the tip and tilt of the wavefront at each sub-aperture. The global tip and tilt aberrations, given by the average horizontal and vertical centre of mass shifts, are removed. Multiplication of a vector comprising these spot motions with the control matrix yields the necessary changes to the actuator values to restore the null wavefront, with these changes being integrated over time. Where actuators fall just outside the pupil their voltages are set to the average of their nearest neighbours. The loop gain is set automatically using a measure of closed-loop stability that is based on the centroid position variance over two seconds, with a typical gain being 0.3.

System aberrations, including non-common path errors, cause a spread in the area of retina illuminated and a reduction in the amount of light focused through the pinhole, reducing the signal-to-noise ratio (SNR). Such aberrations are compensated using an image optimization protocol. The 2.4× scale model eye was placed in the system and the residual system aberrations were compensated with the deformable mirror using the Nelder-Mead simplex algorithm (Nelder & Mead, 1965) to find the actuator voltages necessary to optimize the output at the pinhole via one of two methods. The first method used a CCD placed directly in the focal plane of the AOSLO, where the pinhole is normally located, and maximizes the sharpness of the PSF, which we define as

$$ϵ = \frac{Σ {I ( x , y )}^{2}}{{( Σ I ( x , y ) )}^{2}}$$

where I(x,y) is the intensity of pixel (x,y). The second method used the raw image from the AOSLO collected through the pinhole and minimizes the inverse of the total intensity in the image, maximizing the throughput of the pinhole. The wavefront centroid positions are measured whilst this mirror vector is applied and are used as the null reference positions to which the closed loop attempts to converge.

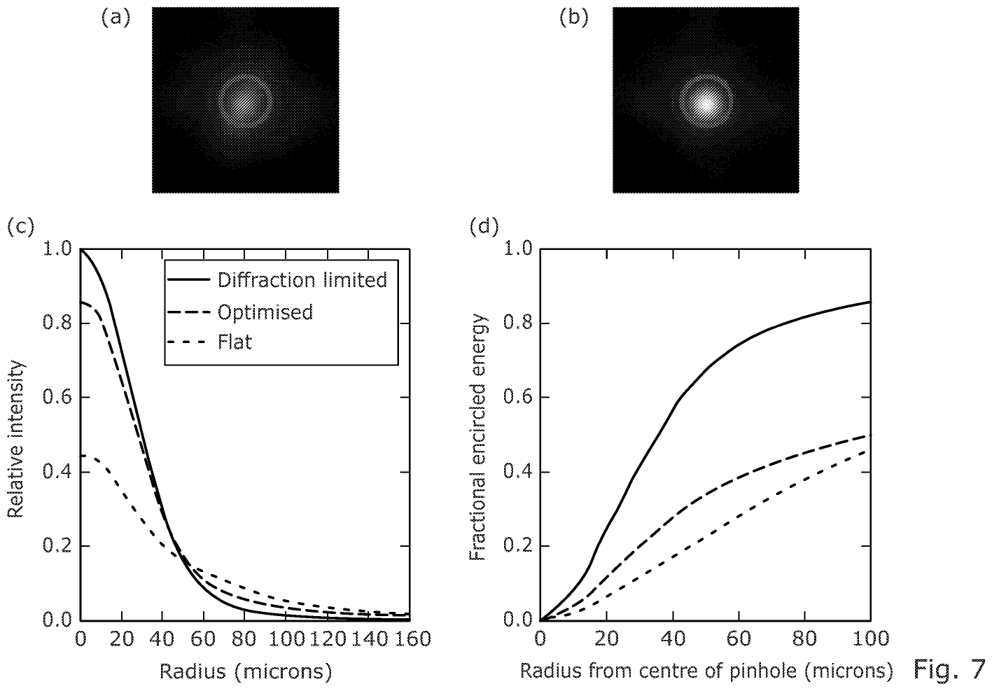

FIG. 7 shows the PSF at the pinhole generated with (a) a flat (zero voltage) DM and (b) the optimum mirror shape determined by maximizing the PSF sharpness. The circle indicates the Airy diameter for light of wavelength 850 nm for a 7 mm pupil diameter at the eye. The improvement in (c) the PSF and (d) the improvement throughput (encircled energy) are shown and the optimized PSF is compared to that expected from the Zemax model (diffraction-limited).

Data Capture

In an AOSLO, retinal images are reconstructed based on the scan position and the signal from the imaging detector. The embodiment shown uses an off-the-shelf digital oscilloscope (such as the Picoscope 3403B, Picotech) to record these signals, which are streamed to the control PC in real-time. Other than a 20 MHz bandwidth filter included in the oscilloscope, all signal processing is done in software. The maximum data rate of the oscilloscope over USB 3.0 is 125 MSamples/s shared between all three channels. The scanning system has 533 lines (forward and reverse) per frame and a frame rate of 30 Hz. This allows the system to capture up to 2605 intensity samples per scan line, 1302 per direction. The desired field of view of the system is 1° across a scan line and, to achieve Nyquist sampling, the pixel scale should be half of the lateral resolution, i.e. 13 arcseconds (1 μm on the retina), giving a minimum number of pixels per scan line of 277 in each direction. We are therefore not limited by the data rate of the oscilloscope and we are able to oversample (and average) by a factor of up to 4.7. The digital oscilloscope has a discrete number of available sampling intervals, we chose a sampling interval of 24 ns, which allowed the highest possible integer oversampling rate (four). To account for the non-integer number of pixels per scan line we perform a 1D interpolation on the raw intensity data before downsampling by a factor of four via averaging.

Image Reconstruction

Data from the digital oscilloscope is received as a continuous stream and individual frames are separated using a software trigger that is based on the scan position, which uses hysteresis to improve the triggering accuracy. There is a small time difference between the mechanical motion of the scanner and its monitoring signal that must be accounted for. Therefore, fine-tuning of the software trigger is performed in post-processing by comparing the forward and reverse scan intensity data, which should be almost identical except for noise.

The scan pattern of the AOSLO is non-linear and so captured intensity data must be linearized using the scan position data. The horizontal, fast scan is produced by a resonant scanner with a sinusoidal pattern and the vertical, slow scan is produced by a galvanometer with an approximately linear pattern (ignoring the fly-back). To reduce noise on the scan position measurement we produce fits to the data (a sinusoidal fit to the horizontal, fast scan data and a second order polynomial fit to the vertical, slow scan data) and this is used as a look-up table to produce linear spatial sampling.

For display purposes during the imaging session, only coarse triggering and non-integer pixel compensation are performed, allowing a live video to be viewed at close to real-time. Fine-tuning of the software trigger and linearizing the data is performed on a frame-by-frame basis in post processing, which accounts for any changes in the scan pattern during the imaging session. The result of these data processing steps is a pair (forward and reverse scan) of 480×330 pixel images of a 1.6°×1° patch of the retina. The size of the scan patch is limited by the mechanical range of the scanner and not by image quality since diffraction-limited imaging is theoretically possible over 2°. Typically, these two images are additionally averaged to further suppress noise, such that each pixel in the final image is effectively an average of eight samples (single forward or reverse frames are oversampled and averaged by a factor of four). We store all the raw data from the oscilloscope allowing us to resample and re-process the images as necessary. The final image can also be composed by interleaving the forward and reverse scan lines and by maintaining an oversampling factor of two, giving a 960×660 pixel image of the same patch of retina, in which each pixel is an average of two samples.

Imaging Protocol

Imaging and wavefront sensing is carried out, in one embodiment, using an 850 nm (50 nm FWHM) superluminescent diode (Superlum). The AOSLO imaging path is directed to the eye via reflection from a dichroic filter, allowing visible wavelengths (<800 nm) to pass through. A display is located at the end of the bench 1.75 m from the eye and in a plane that is conjugate to the focal plane of the AOSLO. We generally avoid using cycloplegic eye drops where possible, allowing participants to focus naturally on the display, and the selection of a long imaging wavelength significantly reduces the impact of the illumination on pupil dilation. Participants are positioned on an adjustable bite-bar to maintain eye-position stability and the eye is aligned to the system using the wavefront-sensor image as a guide. Trial lenses are used to correct for large refractive errors, or to induce a focus shift for viewing different retinal layers.

Retinal locations are targeted by directing the participant's fixation via a target on the display, assuming foveal fixation in normal eyes. Where appropriate, in patients with diseased retina, we adjust the relative position of the target according to their preferred retinal locus and use features in their fundus and OCT images to check accuracy. Light collection is performed using either an avalanche photodiode (APD410A, Thorlabs) or photomultiplier tube (H7422-50, Hamamatsu), depending on the sensitivity required. The minimum voltage range on the digital oscilloscope is ±50 mV, so when using the avalanche photodiode with very low signal levels an additional (10 times) voltage amplifier is required.

It will of course be appreciated that other imaging protocols, and different equipment, may be used.

It will be appreciated that embodiments of the invention can provide an AOSLO with a small footprint, simplified alignment and low-cost hardware interfaces. The embodiment of FIG. 1 has been shown to achieve diffraction limited imaging in healthy retina and has been applied to imaging in patients with inherited retinal degeneration, including 16 patients and 19 healthy controls to date.

1. A scanning laser ophthalmoscope comprising: a light source arranged to generate a beam of light; a deformable mirror relay; a first scanning relay; a second scanning relay; a wavefront sensor configured to detect distortions; and a detector; wherein:
the deformable mirror relay comprises a deformable flat mirror configured to compensate for the distortions detected by the wavefront sensor;
each of the scanning relays comprises a flat scanning mirror configured to scan the beam across an eye of a subject; and
at least one of the relays has an entry focal plane and an exit focal plane and further comprises a spherical mirror arranged to reflect the beam from the entry focal plane onto the flat mirror and to reflect light from the flat mirror into the exit focal plane.

2. A scanning laser ophthalmoscope according to claim 1 wherein said one of the relays is the deformable mirror relay.

3. A scanning laser ophthalmoscope according to claim 2 wherein the relay further comprises at least one fold mirror between the spherical mirror and at least one of the entry focal plane and the exit focal plane, whereby at least one of the entry focal plane and the exit focal plane is not coplanar with the deformable mirror.

4. A scanning laser ophthalmoscope according to claim 1 wherein said one of the relays is one of the scanning relays.

5. A scanning laser ophthalmoscope according to claim 1 wherein each of the three relays has an entry focal plane and an exit focal plane and further comprises a spherical mirror arranged to reflect the beam from the respective entry focal plane onto the respective flat mirror and to reflect light from the respective flat mirror into the respective exit focal plane.

6. A scanning laser ophthalmoscope according to claim 5 wherein the three relays are coplanar.

7. A scanning laser ophthalmoscope according to claim 1 further comprising a light source beam splitter arranged to direct at least some of the light generated by the light source towards the relays, and to direct at least some of the light from the relays towards the detector.

8. A scanning laser ophthalmoscope according to claim 1 further comprising a wavefront sensing beam splitter arranged to direct at least some of the light from the relays towards the wavefront sensor, and to direct at least some of the light from the wavefront sensor towards the detector.

9. A scanning laser ophthalmoscope according to claim 1 wherein at least two of the relays, are arranged in respective modules which are configured to be assembled together for use.

10. A scanning laser ophthalmoscope according to claim 9 wherein the modules are configured to be assembled in a plurality of different operable configurations.

11. A scanning laser ophthalmoscope according to claim 9 wherein each of the modules has an entrance focal plane and an exit focal plane, and the ophthalmoscope includes connecting means arranged to connect the modules together so that one of the focal planes of one of the modules is coincident with one of the focal planes of the other of the modules.

12. A scanning laser ophthalmoscope according to claim 10 wherein each of the modules has an entrance focal plane and an exit focal plane, and the ophthalmoscope includes connecting means arranged to connect the modules together so that one of the focal planes of one of the modules is coincident with one of the focal planes of the other of the modules.
